(19) United States
(12) Patent Application Publication
(10) Pub. No.: US 2004/0019387 A1
(43) Pub. Date: Jan. 29, 2004

(54) Laryngotracheal devices and methods of use thereof

(76) Inventors: Philippe Monnier, Lausanne (CH); Rene Gilliard, Saint-Sulpice (CH)

Correspondence Address: Ivor R. Elrifi, Mintz, Levin, Cohn, Ferris,, Glovsky and Popeo, P.C., One Financial Center, Boston, MA, 02111, US

(21) Appl. No.: 10/440,785

(22) Filed: May 19, 2003

Related U.S. Application Data
(60) Provisional application No. 60/381,939, filed on May 20, 2002.

(51) Int. Cl. A61F 2/04

(52) U.S. Cl. 623/23.64; 623/23.7; 623/902

(57) ABSTRACT

The present invention relates to laryngotracheal devices and methods of use thereof in surgical procedures. In particular, the invention relates to the uses of laryngotracheal devices to treat subglottic stenoses.

### RELATED APPLICATIONS

This application claims the benefit of U.S. Ser. No. 60/381,939, filed May 20, 2002, which is incorporated herein by reference in its entirety.

### FIELD OF THE INVENTION

The present invention relates generally to laryngotracheal devices useful in surgical procedures and to methods of using such laryngotracheal devices. In particular, the invention relates to methods of using such devices to manage laryngotracheal stenoses.

### BACKGROUND OF THE INVENTION

Laryngotracheal stenosis, such as subglottic stenosis (SGS), is a complication of medical procedures, including prolonged endotracheal intubation, as can also result from external laryngeal trauma (e.g., from by motor vehicle accidents and assault), infections, or congenital abnormalities. Subglottic stenosis is of particular importance in infants and children, since the relatively small size of the airway, combined with the subglottis being the narrowest point of the pediatric airway, predisposes infants and children to the condition, wherein small amounts of scarring result in significant airway obstruction.

Subglottic stenosis can be congenital or acquired. The congenital form is related to inadequate recanalization of the laryngeal lumen after completion of the normal epithelial fusion at the end of the third month of gestation. It can be membranous or cartilaginous. The membranous type often involves the true vocal cords. The cartilaginous type is usually shelf-like at the level of the cricoid and easy to diagnose endoscopically. In other instances, the subglottic appearance is normal and only after “sizing” the airway can the appropriate diagnosis be made. The cartilaginous variety of subglottic stenosis is very rarely managed successfully with dilation or with laser techniques.

Acquired subglottic stenosis in infants is often related to prolonged endotracheal intubation, and has a frequency of 1-8%. The most commonly affected area in children is the cricoid. A multitude of factors (i.e., small cricoid, reflux, infection, and tube movement and replacement) are important in predisposing infants to acquired subglottic stenosis.

Laryngotracheal stenosis can be treated by surgery and stenting of the airway. Commercially available stents include the Aboulker's stent, Eliachar's laryngotracheal stent, and the Montgomery T-tube, all of which are generally cylindrical in shape. However, the glottis and upper larynx are non-cylindrical and more triangular in shape with flattened sides. Therefore, cylindrical stents are deficient in their ability to maximize healing without inducing supraglottic ulcerations, granulation tissue formation and recurrent scarring. Although Eliachar's laryngotracheal stent is non-cylindrical, its general shape does not precisely conform to the inner laryngeal contours of a human larynx; furthermore, it only exists in 2 sizes (adult, male and female).

### SUMMARY OF THE INVENTION

In one aspect, the present invention relates to a medical device for placement within a portion of a mammalian patient, the device including a tubular member 1 formed from a substantially rigid material having a hardness of between approximately 30° Shore-A and approximately 70° Shore-A, where the tubular member 1 has a distal end 3a or 3b and a proximal end 2a or 2b and extending longitudinally there between, forming a lumen there through, wherein the tubular member 1 is substantially non-cylindrical at the proximal end. In some embodiments of the invention, the substantially rigid material has a hardness of between approximately 40° Shore-A and approximately 60° Shore-A, approximately 45° Shore-A and approximately 55° Shore-A, or approximately 50° Shore-A. In other embodiments, the substantially rigid material is silicone. The proximal end of the medical device may be either open 2a or closed 2b.

In embodiments of the present invention, the tubular member 1 includes a first portion including the proximal end of the tubular member 1, a second portion including the distal end of the tubular member 1, and a connecting bend 5 formed at a junction of the first portion and the second portion, wherein the proximal end is substantially non-cylindrical and wherein the connecting bend 5 forms an oblique angle between the first portion and the second portion, the connecting bend 5 being closer to the proximal end of the tubular member 1 relative to the distal end of the tubular member 1. This oblique angle is between about 90 degrees and about 180 degrees. In certain embodiments, the angle is between about 120 degrees and about 160 degrees. In a specific embodiment of the invention the angle is about 130 degrees. In another specific embodiment of the invention the angle is about 155 degrees.

In embodiments of the invention the tubular member 1 has an outer diameter between about 3 mm and about 20 mm, e.g., about 6 mm, about 7 mm, about 8 mm, about 9 mm, about 10 mm, about 11 mm, about 12 mm, about 13 mm, about 14 mm, or about 15 mm.

In some embodiments of the invention, the medical device also includes a substantially L-shaped tracheotomy connector member 13 that is operably connected to the tubular member 1.

In other embodiments of the invention, the medical device also includes a fixation member 12 that is substantially flexible and operably connected to the tubular member 1. In some embodiments, the fixation member 12 is an inner silicone tongue. In other embodiments, the fixation member 12 is an outer silicone tongue.

In some embodiments of the present invention, the proximal end of the tubular member 1 has a larger outer diameter than the distal end of the tubular member 1.

In further embodiments, the medical device also includes a substance capable of being released in a controlled manner from the device, such as a polypeptide growth factor, a hormone, an anti-inflammatory agent, an anti-scar formation compound, or an anti-microbial agent.

In some embodiments of the invention, the shape of the medical device is substantially similar to the inner laryngotracheal contours of a human. In related embodiments, the device is formed in the shape of a human larynx.

Another aspect of the present invention relates to methods of treating a laryngotracheal stenosis, by endoscopically inserting a medical device into the larynx of a mammalian patient suffering from laryngotracheal stenosis, where the medical device includes a tubular member 1 formed from a substantially rigid material having a hardness of between approximately 30° Shore-A and approximately 70° Shore-A, the tubular member 1 having a distal end 3b and a proximal end 2b and extending longitudinally there between, forming a lumen there through, wherein the tubular member 1 includes a first portion comprising the proximal end 2b of the tubular member 1, a second portion including the distal end 3b of the tubular member 1, and a connecting bend 5 formed at a junction of the first portion and the second portion, wherein the proximal end 2b is substantially non-cylindrical and, wherein the connecting bend 5 forms an oblique angle between the first portion and the second portion, the connecting bend 5 being closer to the proximal end 2b of the tubular member 1 relative to the distal end 3b of the tubular member 1, such that the connecting bend 5 of the tubular member 1 contacts the arytenoideus cartilage of the patient, thus maintaining the appropriate interarytenoid distance, such that the laryngotracheal stenosis is treated upon insertion.

In embodiments of the invention, the laryngotracheal stenosis is a supraglottic, glottic, subglottic or upper tracheal stenosis. The proximal end of the tubular member 1 may be open or closed.

In some embodiments, the medical device further includes a substantially flexible fixation member 12, wherein the fixation member 12 has a proximal end and a distal end, the proximal end being operably connected to the tubular member 1, and a substantially L-shaped tracheotomy connector member 13, whereby the method also includes drawing the distal end of the fixation member 12 through a tracheostoma 10 of the patient, fixing the distal end to a fixation means, and operably connecting the connector member 12 to the tubular member 1.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and are not intended to be limiting.

Other features and advantages of the invention will be apparent from the following detailed description and from the claims.

### BRIEF DESCRIPTION OF THE DRAWINGS

FIG. 1 is a photographic image of a preferred embodiment of the present invention. FIGS. 1a and 1c are postero-lateral views; FIG. 1b is a posterior view of the device, and FIGS. 1d-1f are cross-sectional views of the device.

FIG. 2 is a schematic representation of an “open” prosthesis, containing a tubular member 1 inserted into the larynx and trachea of a human. Both the proximal 2a and distal 3a ends of the tubular member 1 are open to allow air influx 4 into the lungs. The tubular member 1 is bent at an angle (e.g., and oblique angle) to form a connecting bend 5 close to the proximal end 2a of the tubular member 1, such that the proximal end 2a of the tubular member 1, which has an outer diameter larger than the distal end 3a, contacts the arytenoideus cartilage 6 of the patient. The tubular member 1 is secured with transcutaneous sutures 7.

FIG. 3 is a schematic representation of a “closed” prosthesis including a tubular member 1 inserted into the larynx and trachea of a human. The proximal end 2b of the tubular member 1 is closed, and distal end 3b of the tubular member 1 is open. The tubular member 1 is bent at an angle (e.g., and oblique angle) to form a connecting bend 5 close to the proximal end 2b of the tubular member 1, such that the proximal end 2b of the tubular member 1, which has an outer diameter larger than the distal end 3b, contacts the arytenoideus cartilage 6 of the patient. The tubular member has an opening 9 of similar size to a tracheostoma 10, and the distal end of the tubular member 1 is cut at an oblique angle 11 just below the opening 9 to be aligned with the tracheostoma 10. Connected to the tubular member 1 is a fixation member (e.g., a silicone “tongue”) 12 which can be drawn through the tracheostoma 10 and reflected cranially and then secured by attaching it to a fixation means such as a cord that encircles the patient's neck. Also connected to the tubular member 1 is a substantially L-shaped tracheotomy connector member 13 having a lumen 14 formed between a proximal end 15 and a distal end 16 thereof. The distal end 16 of the tracheotomy connector 13 is inserted into the prosthesis opening 9 and through the distal end 3b of the tubular member 1.

FIG. 4 is a schematic representation of a “closed” prosthesis providing cross-sectional views of the proximal end 2b of the device that demonstrate the non-tubular shape of the proximal end 2b.

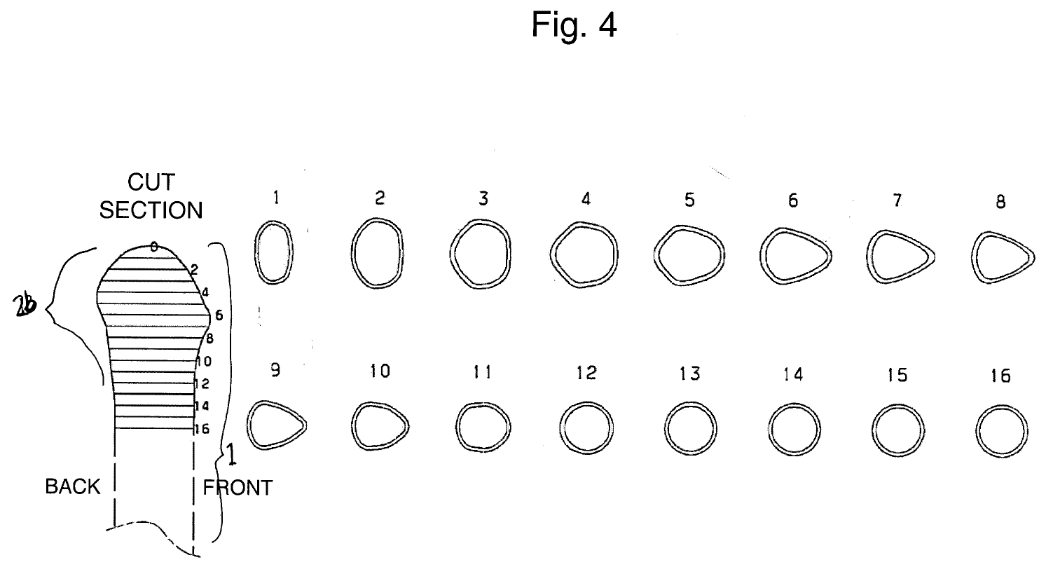

FIG. 5 is a photographic image of a collection of prostheses of varying diameters.

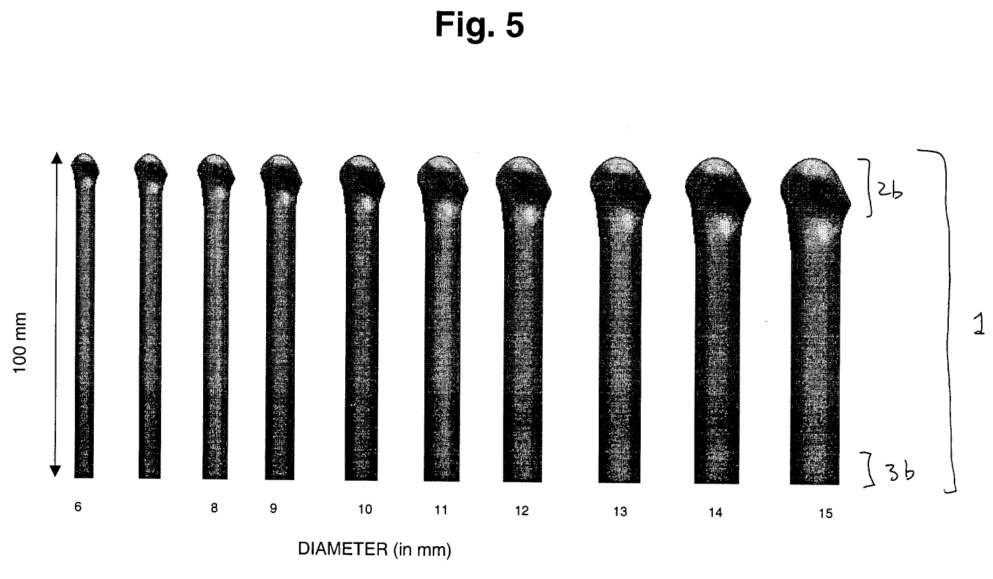

FIG. 6. is a series of schematic images of two embodiments of the present invention. FIG. 6a is a lateral view of a “closed” prosthesis having a suitable length and outer diameter for use in an adult human. FIG. 6b is a cross-sectional view of the device depicted in FIG. 6a. FIGS. 6c and 6d are lateral views of a “closed” prosthesis having a suitable length and outer diameter for use in a pre-pubescent human.

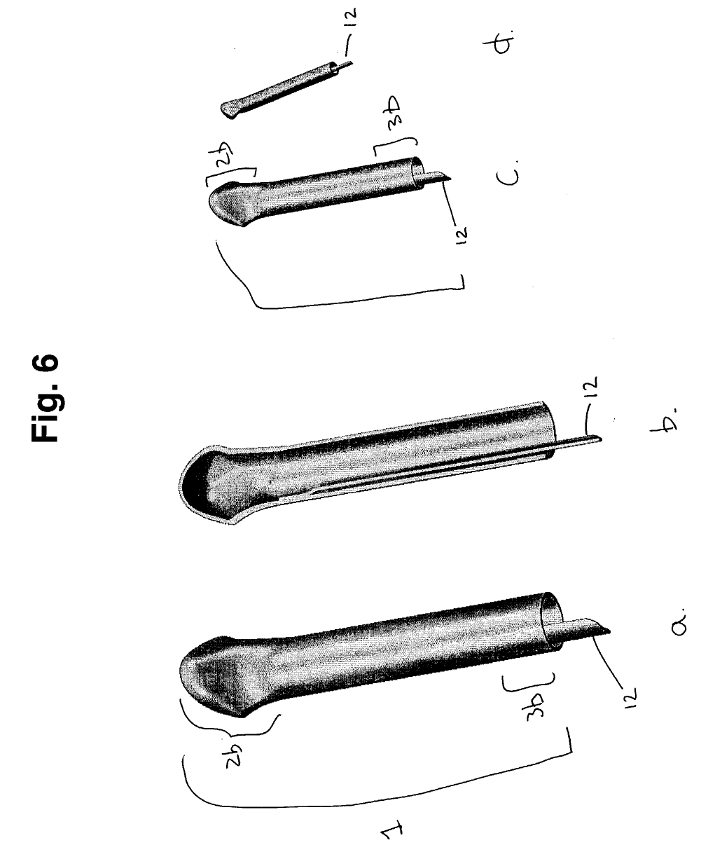

FIG. 7 is a photographic image of a collection of views of the proximal end 2b of a “closed” prosthesis of the present invention, which demonstrate the non-tubular nature of the proximal end 2b.

FIG. 8 is a photographic image a preferred embodiment of the present invention, which shows a proximal end 2b and connecting bend 5 of a “closed” prosthesis, and the angles formed by the intersection of the proximal end 2b and the distal end 3b of the tubular member 1.

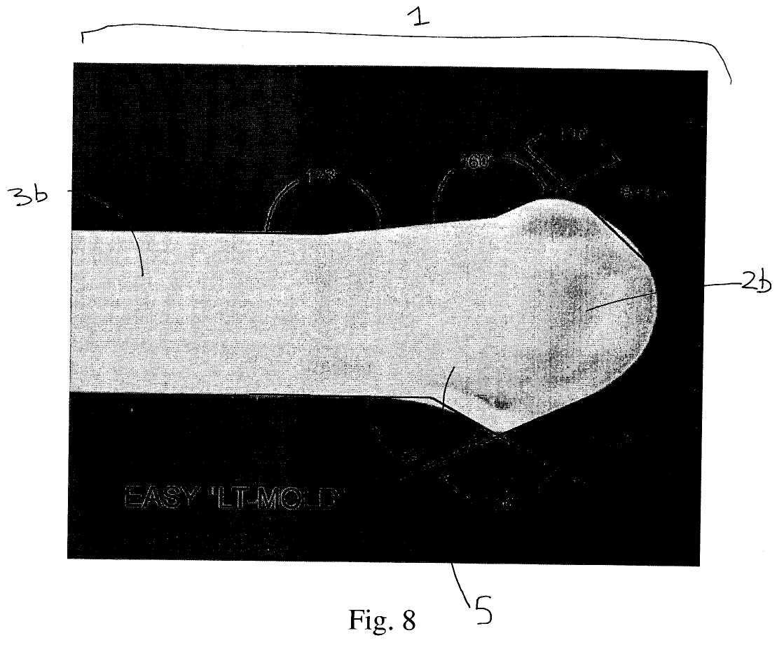

### DETAILED DESCRIPTION OF THE INVENTION

In view of the negative impact of laryngotracheal stenoses on patient recovery, it would be desirable to have improved devices for managing laryngotracheal stenosis during a surgical procedure and during the healing process.

The present invention is directed in part to apparatuses and methods for reversing laryngotracheal stenoses, such as supraglottic, glottic, subglottic or upper tracheal stenosis. While the present invention is described in detail as applied to laryngotracheal stenoses, those skilled in the art will appreciate that the present invention can be applied to other surgical procedures and other internal organs where locally preserving the lumen of a tissue is a primary goal (e.g., coronary arteries, bile ducts, the urethra, and the esophagus).

“Hardness” as used herein can be determined with a durometer, such as a Shore A durometer at 20° centigrade. If the durometer indenter completely penetrates the sample, a reading of 0 is obtained. If no penetration occurs, a reading of 100 results. Hardness can also be determined by one of ordinary skill in the art using electrical resistivity.

Open Prostheses.

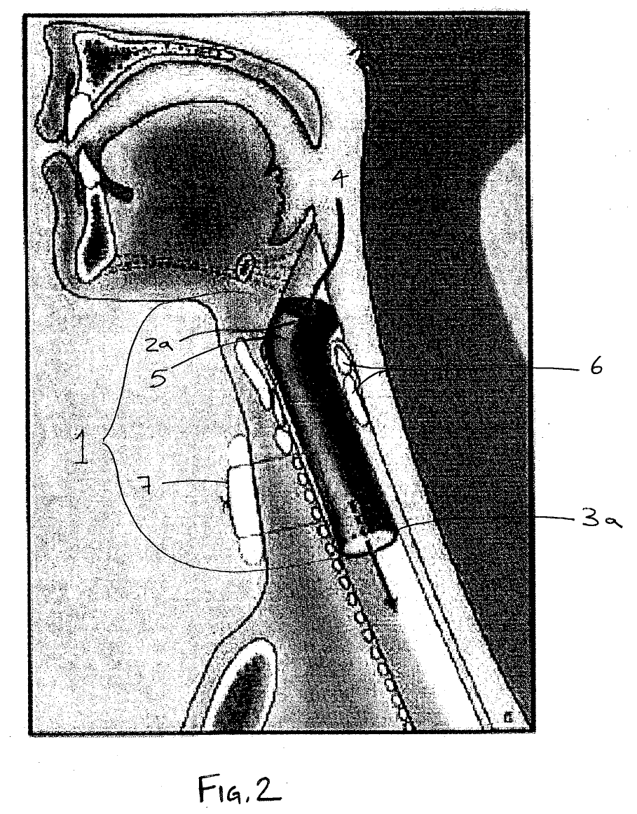

A first preferred embodiment of the present invention includes a medical device termed an “open” prosthesis. FIG. 2 is a schematic representation of an “open” prosthesis, containing a tubular member 1 inserted into the larynx and trachea of a human. Both the proximal 2a and distal 3a ends of the tubular member 1 are open to allow air influx 4 into the lungs. The tubular member 1 is bent at an angle (e.g., and oblique angle) to form a connecting bend 5 close to the proximal end 2a of the tubular member 1, such that the proximal end 2a of the tubular member 1, which has an outer diameter larger than the distal end 3a, contacts the arytenoideus cartilages 6 of the patient. The tubular member 1 is secured with transcutaneous sutures 7.

The tubular member 1 is generally formed from a substantially rigid material having a hardness of between approximately 30° Shore-A and approximately 70° Shore-A. The desired hardness of the tubular member 1 will vary with the tissue to be contacted. Also, the desired hardness of the tubular member 1 will vary with the needs of a patient, such as an infant or child benefiting from a tubular member 1 formed from a material softer than 50° Shore-A. It is preferred that the tubular member 1 is sufficiently soft as to avoid or minimize pressure necrosis, as occurs at the medial aspect of the artenoid cartilage. By way of non-limiting example, a medical device suitable for use with infants or children, such as a device with an external diameter of about 8 mm or less, might have a hardness of 30° Shore-A or less, while a medical device suitable for use with adults, or larger children, such as a device with an external diameter of about 9 mm or more, might have a hardness of up to about 70° Shore-A or more. Preferred materials include silicone, polyurethanes, and silicone-urethane copolymers. One preferred tubular member 1 is formed from silicone having a hardness of about 50° Shore-A. In some embodiments, the tubular member 1 is created by molding cadaver larynges and by increasing the interarytenoid distances to obtain the intralaryngeal contours of a fully abducted larynx.

In embodiments of the invention, the tubular member 1 is straight. Alternatively, the tubular member 1 can be bent or curved. For example, the tubular member 1 can have a first portion that includes the proximal end 2b of the tubular member 1, a second portion that includes the distal end of the tubular member 1, and a connecting bend 5, which is contained in the first portion of the tubular member 1 and is generally closer to the proximal end 2a of the tubular member 1 relative to the distal end 3a of the tubular member 1. The connecting bend 5 forms an angle (e.g., an oblique angle) between the first portion and the second portion. The angle of the connecting bend 5 is generally between about 90 degrees and about 180 degrees, preferably between about 120 degrees and about 160 degrees, and more preferably about 130 degrees or about 155 degrees.

The tubular member 1 can be formed from a single piece of a substantially rigid material using methods known to one of ordinary skill in the art, e.g., extrusion or molding.

In the open prosthesis, both the proximal 2a and distal 3a ends of the tubular member 1 are open to allow air influx 4 into the lungs. The size of the opening at each end can be varied to suit the needs of the application, such as to minimize inhalation of fluids and food articles, and to prevent the blockage of the tubular member 1 with secretions.

The proximal end 2a of the tubular member 1 will have an outer diameter larger than the outer diameter of the distal end 3a, such that the position of the tubular member 1 is capable of being fixed relative to the larynx. The outer diameter of the distal end 3a of the tubular member 1 is between about 3 mm and about 20 mm, and the outer diameter of the proximal end of the tubular member 1 can be greater than 20 mm. Preferably, the outer diameter of the distal end of the tubular member 1 is between about 6 mm and about 15 mm, and more preferably about 6 mm, about 7 mm, about 8 mm, about 9 mm, about 10 mm, about 11 mm, about 12 mm, about 13 mm, about 14 mm, or about 15 mm.

The open prosthesis is generally non-cylindrical at its proximal end 2a. In some embodiments it is substantially triangular in shape, with flattened sides. The shape of the open prosthesis may be gender-specific. It is well known that prior to puberty, there is no marked difference between the larynx of the male and that of the female. Therefore, in infants and small, pre-pubescent children, an open prosthesis of the invention should be equally useful to both genders. In post-pubescent males, the cartilages are enlarged and the glottis is approximately doubled in size, while in post-pubescent females the change to the larynx is slight. The shape of the prosthesis can also be modified to account for malignancies or benign growths within the laryngotracheal region.

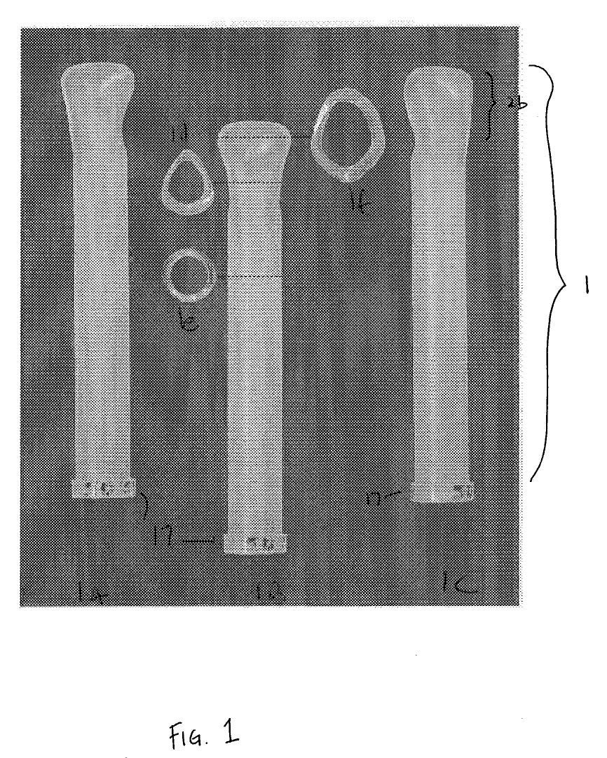

It is advantageous for the tubular member 1 not to move or slide relative to the patient's trachea. The proximal end 2a of the tubular member 1 generally has an outer diameter larger than the distal end 3a, and is shaped such that the tubular member 1 does not move in a caudal direction down the trachea once it is positioned endoscopically. (See FIGS. 1a and 1b). The tubular member 1 can be bent at an oblique angle to form a connecting bend 5, which is in close proximity or contact with the arytenoideus cartilage 6 of the patient. This position, above the cricoid cartilage, reduces the risk that the tubular member 1 will migrate caudally in the trachea. Optionally, the tubular member 1 is secured with one or more transcutaneous sutures 7, such as is achieved by using a Lichtenberger needle holder in a suspension microlaryngoscopic procedure.

In some embodiments, the prosthesis contains one or more anti-microbial agents to reduce or prevent infections. As used herein, the term “anti-microbial agent” includes antibiotics, antiseptics, disinfectants and other synthetic moieties, and combinations thereof, that are soluble in organic solvents such as alcohols, ketones, ethers, aldehydes, acetonitrile, acetic acid, formic acid, methylene chloride and chloroform. Classes of antibiotics that can possibly be used include tetracyclines (i.e., minocycline), rifamycins (i.e. rifampin), macrolides (i.e. erythromycin), penicillins (i.e., nafcillin), cephalosporins (i.e. cefazolin), other beta-lactam antibiotics (i.e. imipenem, aztreonam), aminoglycosides (i.e. gentamicin), chloramphenicol, sulfonamides (i.e., sulfamethoxazole), glycopeptides (i.e., vancomycin), quinolones (i.e., ciprofloxacin), fusidic acid, trimethoprim, metronidazole, clindamycin, mupirocin, polyenes (i.e., amphotericin B), azoles (i.e., fluconazole) and beta-lactam inhibitors (i.e., sulbactam).

Examples of specific antibiotics that can be used include, e.g., minocycline, rifampin, erythromycin, nafcillin, cefazolin, imipenem, aztreonam, gentamicin, sulfamethoxazole, vancomycin, ciprofloxacin, trimethoprim, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, enoxacin, fleroxacin, temofloxacin, tosufloxacin, clinafloxacin, sulbactam, clavulanic acid, amphotericin B, fluconazole, itraconazole, ketoconazole, and nystatin. Other examples of antibiotics, including those listed in U.S. Pat. No. 4,642,104, herein incorporated by reference, will readily suggest themselves to those of ordinary skill in the art.

Examples of suitable antiseptics and disinfectants will readily suggest themselves to those of ordinary skill in the art.

In other embodiments of the present invention, the prosthesis contains one or more therapeutic agent. The therapeutic agent of the invention can be a vasoactive agent, an anti-proliferative agent, an anti-inflammatory agent, an immunomodulating agent, an anti-angiogenic agent, a myocyte growth factor, an anti-viral agent, an anti-parasitic agent, or an anti-tumor agent.

Those skilled in the art will recognize that the prostheses of the invention can contain any other therapeutic agents. For example the prostheses may contain an anti-apoptotic agent, a thrombolytic agent, a pro-angiogenic agent, a contractility improving agent, a complement blocker, an inhibitor of reperfusion injury, a calcium channel blocker, a vasoactive agent, an anti-thrombotic agent, an anti-platelet agent, anti-proliferative agent, an anti-inflammatory agent, an immunomodulating agent, an immunosuppressive agent, an inhibitor of reactive oxygen metabolites, an anti-angiogenic agent, a myocyte growth factor, an iron-chelating agent, an anti-integrin agent, a pro-apoptotic agent, an anti-viral agent, an anti-parasitic agent, a free radical scavenger, or an anti-tumor agent, or a biologically active derivative thereof.

Closed prostheses.

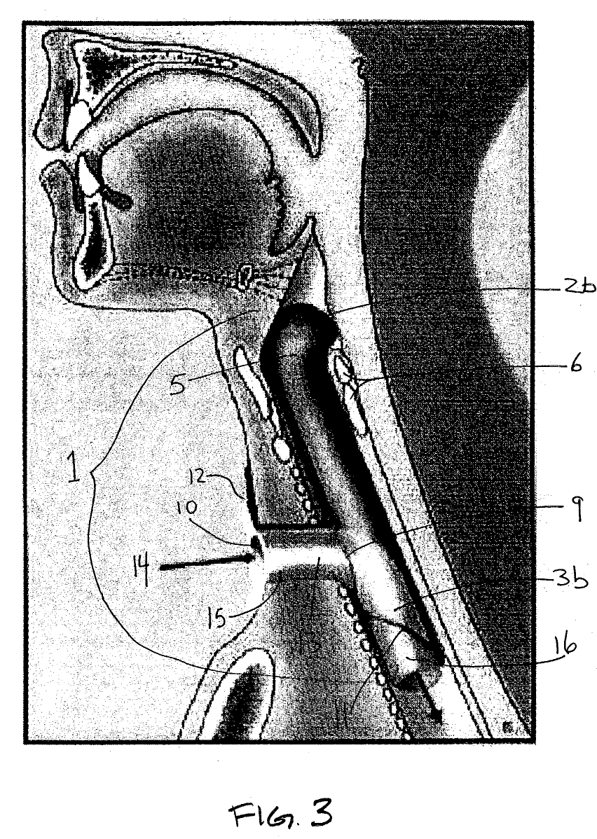

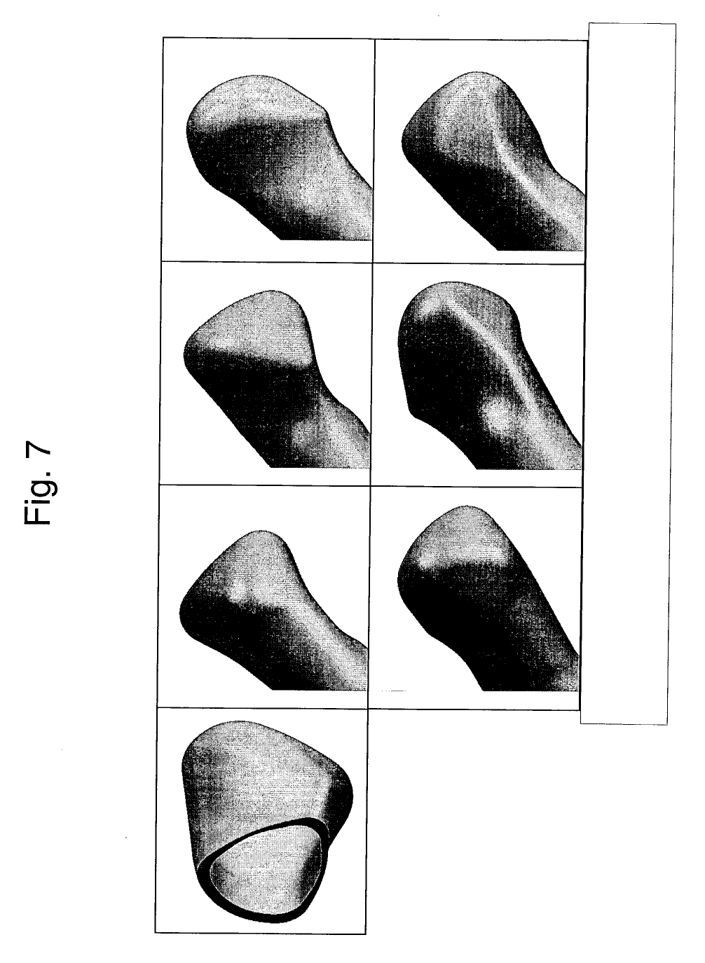

A second preferred embodiment of the present invention includes a medical device termed a “closed” prosthesis. FIG. 3 is a schematic representation of a “closed” prosthesis including a tubular member 1 inserted into the larynx and trachea of a human. The proximal end 2b of the tubular member 1 is closed, and distal end 3b of the tubular member 1 is open. The tubular member 1 is bent at an angle (e.g., and oblique angle) to form a connecting bend 5 close to the proximal end 2b of the tubular member 1, such that the proximal end 2b of the tubular member 1, which has an outer diameter larger than the distal end 3b, contacts the arytenoideus cartilage 6 of the patient. The proximal end 2b is substantially non-cylindrical. An embodiment of the invention is depicted in FIG. 7, which demonstrates the non-tubular nature of the proximal end 2b. The tubular member 1 has an opening 9 of similar size to a tracheostoma 10, and the distal end 3b of the tubular member 1 is cut at an oblique angle 11 just below the opening 9 to be aligned with the tracheostoma 10. Connected to the tubular member 1 is a fixation member, e.g., a silicone “tongue” 12, which can be drawn through the tracheostoma 10, reflected cranially, and then secured by attaching it to a fixation means such as a cord that encircles the patient's neck. Also connected to the tubular member 1 is a substantially L-shaped tracheotomy connector member 13 having a lumen 14 formed between a proximal end 15 and a distal end 16 thereof. The distal end 16 of the tracheotomy connector 13 is inserted into the prosthesis opening 9 and through the distal end 3b of the tubular member 1.

The tubular member 1 is generally formed from a substantially rigid material as described above for the open prostheses.

In embodiments of the invention, the tubular member 1 is straight. Alternatively, the tubular member 1 can be bent or curved, as described above for the open prostheses.

In the closed prosthesis, the proximal end 2b of the tubular member 1 is closed, while the distal end 3b of the tubular member 1 is open. The proximal end 2b may be closed with a permanent or removable cap, seal, or other closing means, or may be formed without a hole. The orientation of the opening at the distal end 3b may be perpendicular to the tubular member 1, or may be on an angle to the tubular member 1, as shown in FIG. 3.

The proximal end 2b of the tubular member 1 will have an outer diameter larger than the outer diameter of the distal end 3b, such that the position of the tubular member 1 is capable of being fixed relative to the larynx. The outer diameter of the distal end of the tubular member 1 is between about 3 mm and about 20 mm, and the outer diameter of the proximal end of the tubular member 1 can be greater than 20 mm. Preferably, the outer diameter of the distal end 3b of the tubular member 1 is between about 6 mm and about 15 mm, and more preferably about 6 mm, about 7 mm, about 8 mm, about 9 mm, about 10 mm, about 11 mm, about 12 mm, about 13 mm, about 14 mm, or about 15 mm.

The closed prosthesis is generally non-cylindrical at its proximal end 2b, and in some embodiments is substantially triangular in shape, with flattened sides, as described above for the open prosthesis.

The closed prosthesis is particularly useful for patients having a tracheostoma, such as a tracheostoma produced by a surgical tracheotomy. In some embodiments, the tubular member 1 has an opening of similar size to a tracheostoma. This opening can be from about 1 to about 20 mm in diameter. Alternatively, an opening can be generated by cutting the tubular member 1 to generate an opening of suitable size. Connected to the tubular member 1 is a fixation member (e.g., a “tongue”) 12 which can be drawn through the tracheostoma and reflected (e.g., cranially, caudally or laterally) and then secured by attaching the fixation member 12 to a fixation means. The fixation member 12 can be made of any flexible material. Preferably, the fixation member 12 is silicone. Suitable fixation means include a cord that encircles the patient's neck. The fixation member 12 is useful to prevent suprastomal collapse, as well as preventing or reducing the formation of gruanulation tissue around the tracheostoma.

In some embodiments, the tubular member 1 of the closed prosthesis is connected to a substantially L-shaped tracheotomy connector member 13 (or, e.g., a “cannula”) having a lumen 14 formed between a proximal end 15 and a distal end 16 thereof. The connector member 13 can by made from any surgically acceptable material. Preferably, the connect member 13 is substantially rigid, such that it can be easily introduced or withdrawn through the tracheostoma and the tubular member 1. The distal end 16 of the tracheotomy connector 13 is inserted into the prosthesis opening 9 and through the distal end 3b of the tubular member 1.

In some embodiments, the closed prosthesis contains one or more anti-microbial agent and/or one or more therapeutic agents, as described above for the open prosthesis.

One use of the closed prosthesis is described in Example 1.

It should be noted that one of ordinary skill in the art would readily be able to convert a closed prosthesis into an open prosthesis by cutting a hole in at or near the proximal end 2b of the tubular member 1. Conversely, one of ordinary skill in the art would be able to convert an open prosthesis into a closed prosthesis by blocking the opening at the proximal end 2b of the tubular member 1, such with a cap or seal.

### EXAMPLES

### Example 1

Use of the Medical Device of the Present Invention to Treat Subglottic Stenosis in a Patient with a Tracheostomy

The medical device of the present invention is useful, in part, to reverse glottic stenosis in a patient with a tracheostomy. In one embodiment the medical device is termed the Easy LT-Mold, and includes a substantially non-cylindrical silicone tubular member 1 (a “prosthesis”) (hardness of approximately 50° Shore-A) having a closed proximal end 2b that is of greater outer diameter than the outer diameter of an open distal end 3b, and also having a silicone fixation member (a “tongue”) 12, that is substantially flexible, which is operably connected to the prosthesis.

Once a tracheostoma has been generated in the patient, the distance between the anterior commissure of the patient's larynx and the upper region of the tracheostoma is measured and marked on the prosthesis. Next, an opening of similar size to the tracheostoma is cut into the prosthesis at the marked distance, and the prosthesis is then rendered a suitable length by cutting off a portion of the distal end 3b of the prosthesis at an oblique angle just below the tracheostoma. The silicone tongue 12 is then drawn through the tracheostoma and reflecting it cranially. The tongue is then secured by attaching it (such as by a clip, pin, or other fixation means) to a cord that encircles the patient's neck. One end of a substantially L-shaped tracheotomy connector member (a “cannula”) 13, having a lumen formed between a proximal and a distal end thereof, is inserted through the tracheostoma and the opening of the prosthesis, so that a substantial portion of the cannula is contained within the trachea of the patient. The tongue maintains the position of the prosthesis while the cannula is being inserted or withdrawn, and also prevents the formation of granulation tissue around the tracheostoma.

### Other Embodiments

It is to be understood that, while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A medical device for placement within a portion of a mammalian patient, the device comprising a tubular member formed from a substantially rigid material having a hardness of between approximately 30° Shore-A and approximately 70° Shore-A, said tubular member having a distal end and a proximal end and extending longitudinally there between, forming a lumen there through, wherein said tubular member is substantially non-cylindrical at said proximal end.

2. The medical device of claim 1, wherein the substantially rigid material has a hardness of between approximately 40° Shore-A and approximately 60° Shore-A.

3. The medical device of claim 1, wherein the substantially rigid material has a hardness of between approximately 45° Shore-A and approximately 55° Shore-A.

4. The medical device of claim 1, wherein the substantially rigid material has a hardness of approximately 50° Shore-A.

5. The medical device of claim 1, wherein said substantially rigid material is silicone.

6. The medical device of claim 1, wherein said proximal end is closed.

7. The medical device of claim 1, wherein said proximal end is open.

8. The medical device of claim 1, wherein said tubular member comprises
a first portion comprising the proximal end of said tubular member,
a second portion comprising the distal end of said tubular member, and
a connecting bend formed at a junction of said first portion and said second portion, wherein said first portion is substantially non-cylindrical and wherein said connecting bend forms an oblique angle between said first portion and said second portion, said connecting bend being closer to the proximal end of said tubular member relative to the distal end of said tubular member.

9. The medical device of claim 8, wherein said angle is between about 90 degrees and about 180 degrees.

10. The medical device of claim 8, wherein said angle is between about 120 degrees and about 150 degrees.

11. The medical device of claim 8, wherein said angle is about 130 degrees.

12. The medical device of claim 1, wherein said tubular member has an outer diameter between about 3 mm and about 20 mm.

13. The medical device of claim 12, wherein said outer diameter is between about 6 mm and about 15 mm.

14. The medical device of claim 12, wherein said outer diameter is selected from the group consisting of about 6 mm, about 7 mm, about 8 mm, about 9 mm, about 10 mm, about 11 mm, about 12 mm, about 13 mm, about 14 mm, and about 15 mm.

15. The medical device of claim 1, further comprising a substantially L-shaped tracheotomy connector member, said connector member being operably connected to said tubular member.

16. The medical device of claim 1, further comprising a fixation member, said fixation member being substantially flexible, said fixation member being operably connected to said tubular member.

17. The medical device of claim 1, wherein said fixation member is a inner silicone tongue.

18. The medical device of claim 1, wherein said proximal end of said tubular member has a larger outer diameter than said distal end of said tubular member.

19. The medical device of claim 1, further comprising a substance capable of being released in a controlled manner from said device, said substance selected from the group consisting of a polypeptide growth factor, a hormone, an anti-inflammatory agent, and an anti-scar formation compound.

20. The medical device of claim 1, further comprising an anti-microbial agent.

21. The medical device of claim 1, wherein said device is substantially similar to the inner laryngotracheal contours of a human.

22. The medical device of claim 1, wherein said device is formed in the shape of a human larynx.

23. A method of treating a laryngotracheal stenosis, comprising:
endoscopically inserting a medical device into the larynx of a mammalian patient suffering therefrom, said medical device comprising a tubular member formed from a substantially rigid material having a hardness of between approximately 30° Shore-A and approximately 70° Shore-A, said tubular member having a distal end and a proximal end and extending longitudinally there between, forming a lumen there through, wherein said tubular member comprises a first portion comprising the proximal end of said tubular member, a second portion comprising the distal end of said tubular member, and a connecting bend formed at a junction of said first portion and said second portion, wherein said first portion is substantially non-cylindrical and, wherein said connecting bend forms an oblique angle between said first portion and said second portion, said connecting bend being closer to the proximal end of said tubular member relative to the distal end of said tubular member, such that said connecting bend of said tubular member contacts the arytenoideus cartilage of said patient, thus maintaining the appropriate interarytenoid distance,
such that the laryngotracheal stenosis is treated upon insertion.

24. The method of claim 23, wherein said laryngotracheal stenosis is a supraglottic, glottic, subglottic or upper tracheal stenosis.

25. The method of claim 23, wherein the proximal end of the tubular member is closed.

26. The method of claim 23, wherein the proximal end of the tubular member is open.

27. The method of claim 23, wherein said medical device further comprises:
a substantially flexible fixation member, wherein said fixation member has a proximal end and a distal end, said proximal end being operably connected to said tubular member, and
a substantially L-shaped tracheotomy connector member,
whereby said method further comprises:
drawing the distal end of said fixation member through a tracheostoma of said patient and fixing said distal end to a fixation means; and
operably connecting said connector member to said tubular member.

28. The medical device of claim 8, wherein said angle is about 155 degrees.
